(19) United States
(12) Patent Application Publication
(10) Pub. No.: US 2019/0304024 A1
(43) Pub. Date: Oct. 3, 2019

(54) DECISION TOOL FOR USE BY INDIVIDUALS IN HEALTHCARE PLAN SELECTION

(71) Applicant: Ceridian HCM, Inc., Minneapolis, MN (US)

(72) Inventors: John Lawrence Colley, Richmond, VA (US); Allen Michael Hatzimanolis, Richmond, VA (US); Timothy Gerard O'Shea, Richmond, VA (US); Aujang S. Abadi, Henrico, VA (US)

(73) Assignee: Ceridian HCM, Inc., Minneapolis, MN (US)

(21) Appl. No.: 16/361,294

(22) Filed: Mar. 22, 2019

Related U.S. Application Data
(60) Provisional application No. 62/648,631, filed on Mar. 27, 2018.

(51) Int. Cl. G06Q 40/08; G06Q 30/02

(52) U.S. Cl. CPC G06Q 40/08; G16H 10/60; G06Q 30/0283

(57) ABSTRACT

A decision support tool is a computerized system for advising individuals who are choosing from available medical insurance plans/options for themselves or their families. The menu of available options might include as few as two plans as determined by the individual's employer, or it might include scores of plans on a direct-to-carrier basis or through a public or private exchange. The option that is best for one person or family is not best for all. Optimization for an individual or family is a function of likely medical consumption, the detailed benefit designs of the available plans, the individual's contribution to premium, and any employer contribution to an HSA, HRA or FSA (if applicable). The decision-support tool ranks available options from best-fit to worst-fit, and it quantifies the relative values via one Value Score per product.

This application claims the benefit of filing of U.S. Provisional Application No. 62/648631, filed Mar. 27, 2018, which is incorporated by reference herein in its entirety.

The present invention is a tool that helps individual consumers and their families identify the best choice among health insurance plans available to them. The tool processes a range of information to calculate and present a single value score comparison of available health insurance options.

### BACKGROUND

Studies show that without expert guidance, most people make Medical Insurance choices that are no better than random. Ideally, individual consumers and their families would understand what they are likely to receive in benefits in return for what they are likely to incur in costs. Unfortunately, medical costs are very difficult to predict, and benefit designs are bewilderingly complex. Even an industry expert cannot typically make an optimal choice without computerized assistance.

Benefits can be broken into two components: the monetary value of medical goods and services that an individual consumer and their family will consume, and the monetary value of any employer contribution to an HSA, HRA, or FSA (if applicable). An employer's contribution to HSA, HRA, or FSA is straight-forward, but likely medical consumption is anything but straight-forward.

Costs to the consumer also can be broken into two components: the individual's contribution to premium and anticipated out-of-pocket (OOP) costs. The former is straight-forward, but OOP cost is extremely complex. First, the individual must have a projection of likely consumption, and they must understand how that consumption is likely to be distributed across the various service types (e.g. office visits, drugs, labs, x-rays, surgery, hospital, etc.) that will have differing patient-pay attributes. Then the individual must impose the applicable benefit design features (e.g. deductible, co-payment, co-insurance, maximums) to the costs as distributed by service type. While a minority of plans might have a comprehensive deductible and a co-insurance percentage applicable to all service types, it is much more common for each service type and service setting to vary in deductible-applicability and patient-pay amount or percentage.

Benefit-design complexity has evolved to a point where optimal plan-choice decisions are in most cases impossible without machine-based decision-support. To provide customers with a degree of insight, some insurance companies have introduced out-of-pocket calculators. These typically require users to enter specific numbers and types of certain services (e.g. office visits and prescription drugs) on a member-by-member basis. Duration of use of these calculators (often 30-40 minutes), tedium, and uncertainty lead to low use rates of the calculators and a high incidence of abandonment. Further, many types of service (e.g. diagnostic tests, laboratory, x-ray, rehabilitation, etc.) are excluded from consideration because of limitations on individual user time and knowledge.

Several commercial decision-support systems have entered the market. Most are of the calculator-type and are subject to limitations described above: they provide an incomplete picture after long and tedious sessions that dampen usage rates. A few commercial systems supplement user inputs with medical and/or prescription drug claim data. This tends to limit the market to employers large enough to control their own data, typically self-funded employer-based groups of at least several hundred members. Further, availability of paid-claim data for analysis generally lags services-incurred dates by 3-6 months, and data for newer employees will be absent or incomplete.

### SUMMARY

Accordingly, it is an object of the present invention to overcome the drawbacks of existing decision support programs and tools by providing a simple-to-use and relatively fast tool that provides an accurate relative value score for medical insurance plans that are available in a specific set of plans offered to an individual user. Compared to other decision-support systems for medical plan selection, unique attributes of the present decision tool enable it to provide better recommendations in much less time, with much higher voluntary usage rates.

In one example, a method of presenting to a user a ranking of available medical insurance options comprising the steps of providing a processor for storing historical data regarding available medical insurance policies and for calculating estimated future medical insurance costs, administratively inputting and storing in the processor information regarding medical and prescription drug costs by event and condition, by percentile and service type and by geographic location, and administratively inputting and storing in the processor group-specific data regarding benefit design data. Next, a user inputs into the processor the user-estimated medical needs. The processor then calculates an estimated cost of goods and services to be consumed by the user, an estimated out-of-pocket cost to the user, and a net benefit to the user, and using the net benefit calculation plus other quality-of-coverage attributes, then calculating and presenting to the user value scores with respect to each medical insurance option available to the user in the group-specific plan. The value scores may be characterized as a single relative value score. The user-estimated medical needs input by the user may not include user-level medical or prescription drug claim data. The net benefit calculation may be the net of two positives including monetary value of covered services and employer contribution to a user HSA/HRA/FSA account, and two negatives including employee contribution to premium and estimated out of pocket cost. The out of pocket estimate may be an apportionment of projected cost over multiple service types. The apportionment may include at least ten service types. The apportionment by service type may be based on the percentile of the user's projected cost. The apportionment may be the projected cost by member within the contract. The percentiles may be based on distributions from individual-only contracts. The percentiles for multi-person contracts may be derived from Monte Carlo simulations. The benefit design model may be based on plan-level data including gatekeeping, out-of-network benefits, and accumulation method; tier-level data including deductibles, maxima, and contributions; and service-level data including out-of-pocket schema and applicability of deductibles and maxima. The out-of-pocket schema may include co-payments, co-insurance, combined co-payments and co-insurance, and greater of co-payment or co-insurance up to a per-service maximum. The value score calculation may include quality attributes that do not affect out-of-pocket cost including catastrophic protection, gatekeeping, case-specific actuarial value, out-of-network coverage, and negative or near-zero net cost. The user input may be based on only four questions asked. The four questions are who is covered, general propensity to consume medical services, anticipated medical events, and anticipated medical conditions. The method may also comprise the step of calculating and displaying to an employer a percentile-based heat map regarding a summary of multiple user contribution strategy and benefit design.

### BRIEF DESCRIPTION OF THE DRAWINGS

FIG. 1 is an overview flowchart of the process described herein that leads to providing value scores to a user of the decision tool.

FIG. 2 is a flowchart that illustrates the process of calculating services cost.

FIG. 3 is a flowchart that illustrates the calculation of out of pocket cost to a user as calculated by the decision tool.

FIG. 4 is a flowchart illustrating the calculation of the value scores as presented to the user who uses the decision tool.

FIGS. 5A-5G illustrate one example of the progression of user interfaces presented to a user of the tool.

### DETAILED DESCRIPTION

The decision-support tool described herein is unique in several ways. The average user experience is between 3 and 4 minutes, which encourages very high use rates. In entirely optional/voluntary settings (i.e. use not required to enroll), more than half of employees offered the decision-support tool will use the tool to its end (receipt of plan rankings, value scores, and recommendations). For multi-person contracts (e.g. family coverage), the user answers questions on a whole-family basis, while other systems typically require member-by-member entries. Precise numerical estimates of services-to-be-incurred are not required. The decision-support tool considers whole episodes-of-care (including diagnostic tests, laboratory, x-ray, rehabilitation, drugs, and all other service types), and apportionment of costs to the various service types is accomplished via a unique percentile-based approach. Compared to other decision-support systems for medical plan selection, unique attributes of the present decision-support tool/system enable it to provide better recommendations in much less time, with much higher voluntary usage rates.

The computer-based decision-support tool incorporates historical data regarding medical insurance policies and available medical and prescription drug costs by event and condition, by percentile and service type and by geographic location. An employer or group-specific data regarding benefit design specifics are also administratively input into the tool. A user then inputs their user-estimated medical needs. The decision-support tool then calculates a net benefit to the user. This is the net of two benefits (projected service value and employer account contribution) and two costs (premium cost and projected out-of-pocket cost). Finally, using the net benefit calculation and other quality-of-coverage attributes, the tool calculates and presents to the user value scores with respect to each medical insurance option available to the user in the group-specific plan. These value scores assist a user in seeing their own personal best options from a plurality of health benefit plan options that the user has.

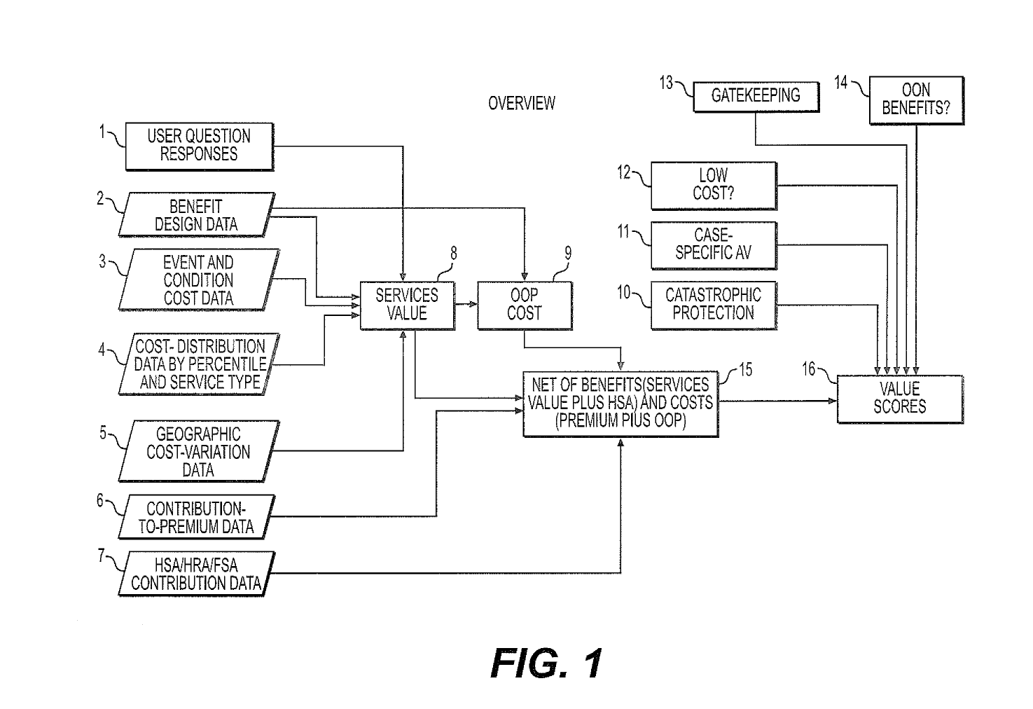

FIG. 1 is an overview of the operation of the decision-support tool and the general steps involved to arrive at the ordinal value scores that are useful for a user to see and consider. After this overview in FIG. 1, some of the detailed steps in the process will be described in connection with the further drawings.

Global Data

The computer-based decision-support system contains data that is previously stored in the tool regarding medical and prescription drug costs by event and condition, by percentile and service type, and by geographic locale at the 3-digit ZIP Code level. These are represented in FIG. 1 as the following:
3) Event and Condition Cost Data
4) Cost-Distribution Data by percentile and service type
5) Geographic Cost-Variation Data

Event and Condition Cost Data 3 are derived empirically from claim data. Cost-Distribution Data by percentile and service type 4 at the individual level are derived empirically from claim data. Percentile distributions for multi-person contracts (Individual plus Spouse, Individual plus one child, Individual plus Children, Individual plus Family) are derived from individual-level distributions using Monte Carlo simulation. Geographic Cost-Variation Data 5 are derived from the Geographic Practice Cost Indices that the US Government uses for Medicare reimbursement.

Group-Specific Data

Administrative data entered into the My Clearview computer-based decision-support system on a group-specific basis are represented in the flowcharts as:
2) Benefit-Design Data
6) Contribution-to-Premium Data
7) HSA/HRA/FSA Contribution Data

Benefit-Design Data Model

Benefit-Design Data 2 are entered at the administrator level for each plan offered to members of a particular group. At the plan level, each product (i.e. “plan”) has a Boolean attribute for presence/absence of “gate-keeping” (referral requirements for services other than primary care), and a Boolean attribute for presence/absence of out-of-network benefits. At the “tier level” (because values differ by “rate tier”, i.e. Individual vs. Individual plus Spouse vs. Individual plus one child vs. Individual plus Children vs. Individual plus Family) deductibles and out-of-pocket maximums are entered for each plan offered to members of a group. At the “service level”, for each included type of service, detailed coverage data are entered. The system can be configured to divide overall cost into any number of subsets, but currently the following 27 categories are used:
Rx-Generic;
Rx-Preferred Brand;
Rx-Non-Preferred Brand;
Rx-Specialty High-Cost;
Preventive Services;
Office Visit-Primary Care;
Office Visit-Specialist;
Lab-OP Facility;
Lab-OP Professional Office;
X-rays (Primary);
X-rays (Specialist);
X-rays (all other);
Imaging-OP Facility;
Imaging-OP Professional;
Emergency Department;
Inpatient Facility;
Ambulatory Surgery-OP Facility;
Ambulatory Surgery-OP Professional;
OP Facility (all other);
OP Professional (all other);
Mental Health-OP Facility;
Mental Health-OP Professional;
Speech Therapy-OP Facility;
Speech Therapy-OP Professional;
Occupational Therapy-OP Facility;
Occupational Therapy-OP Professional;
and
Skilled Nursing Facility.

For each service type, detailed coverage information is entered. The out-of-pocket expense (patient-pay amount) is driven by one of four “schemas”:
Co-insurance
Co-payment
A combination of Co-insurance and Co-payment
The greater of Co-insurance or Co-payment, up to a per-service maximum
   As demanded by the particular “schema”, Co-insurance, Co-payment, and/or per-service maximum amounts are entered. Further, whether or not each service type is subject to either of two deductibles, and whether or not each service type is limited by either of two maximums is entered.

Contribution-to-Premium Data and HSA/HRA/FSA Contribution Data

Contribution-to-Premium Data 6 are entered by rate tier for each plan that is to be offered as an option to members of a group. For employer-based plans, the relevant amount is the payroll deduction, or employee's contribution to premium. The employer's contribution is irrelevant for purposes of My Clearview. However, for coverage that is not employer-based, the whole premium is relevant to the user's perspective. HSA/HRA/FSA Contribution Data 7 are relevant only for employer-based coverage, and only for plans for which the employer contributes.

User Question Responses

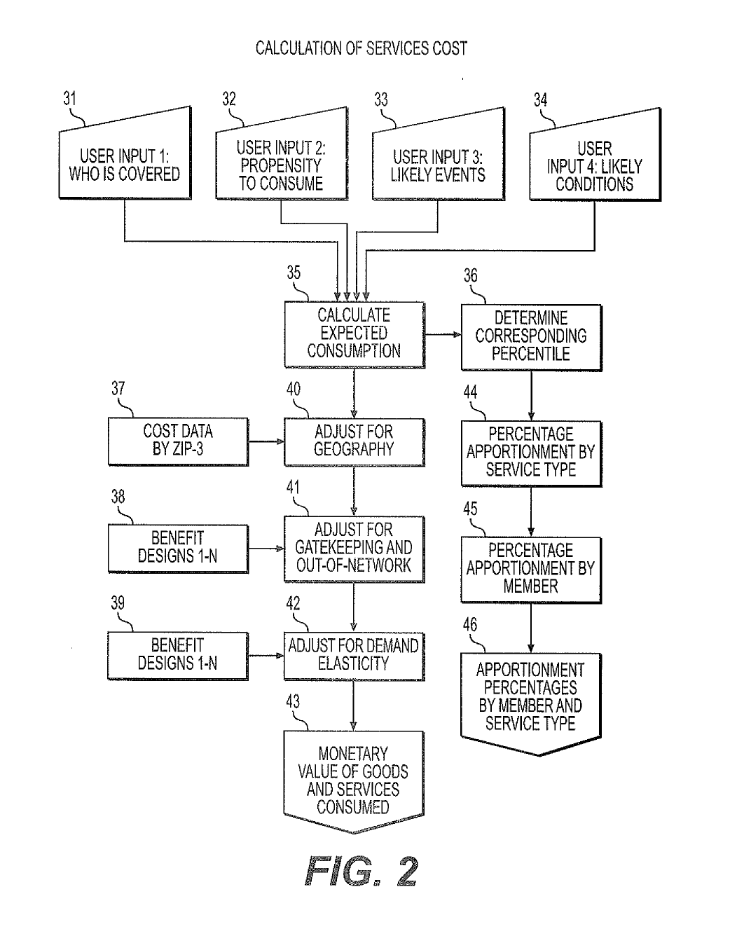

Note that all information above is entered administratively (or acquired electronically via API) upstream of the user experience. After encountering a “landing page” that is customized at the group/employer level, and a listing of available plan options, the user of the present decision tool begins to answer the questions that will lead to recommendations customized to the individual. At the overview level of FIG. 1, the questions are represented in the flowcharts as: 1) User Question Responses. At a more detailed level, and as will be discussed in more detail later, the questions are experienced by the user on four successive screens, represented in the flowcharts (FIG. 2) as:
31) User Input 1: Who is covered?
32) User Input 2: Propensity to consume
33) User Input 3: Likely Events
34) User Input 4: Likely Conditions

Net Benefit

The user answers to the four questions, in combination with Benefit-Design Data 2, Event and Condition Cost Data 3, Cost-Distribution Data by percentile and service type 4, and Geographic Cost-Variation Data 5 lead to an estimate of the expected value/cost of goods and services to be consumed (Services Value 8). In this step, the role of the Benefit-Design Data 2 is to adjust the projected cost for the effects of demand elasticity (people will consume less if their out-of-pocket costs are higher), as well as the effects of “gatekeeping” and out-of-network benefits. In the next step (OOP Cost 9), Benefit-Design Data 2 are applied to the cost estimate in step 8. This leads to the projected out-of-pocket cost 9. Having calculated the expected value of goods and services 8 and the expected out-of-pocket (OOP) cost 9, two of the four components of net benefit 15 are present. The initial ordinal ranking of plans is determined in step 15—Net of benefits (services value 8 plus HSA/HRA/FSA 7) and costs (premium 6 plus OOP 9). Therefore, the net benefit 15 or cost to the consumer is comprised of four components, two of which (in accounting terms) are credits, and two of which are debits:
6) Contribution-to-Premium Data
7) HSA/HRA/FSA Contribution Data
8) Services Value
9) OOP Cost
   Conceptually, as shown, the “net benefit” 15 is what one receives in benefits in return for what one pays. On the credit side, one receives the “covered” goods and services that the individual or family will consume. Also, if applicable for a given option, one might receive an employer contribution to a Health Savings Account (HSA), Health Reimbursement Arrangement (HRA), or Flexible Spending Account (FSA). On the debit side, what one pays to receive these benefits is the sum of premium cost and OOP cost. While the calculation of two components of net benefit 15 (value of services 8 and OOP cost 9) is complex, determination of the other two is straight-forward and unambiguous. The amount of a person's contribution to premium 6 and applicable HSA, HRA, or FSA contribution 9 is a function of plan and rate tier, and these amounts are known a priori.

Value Scores

Once the ordinal rankings of net benefit 15 are determined, each product is assigned a Value Score (#16, Value Scores) on a 100-point scale, higher being better. Modifying factors are:
10) Catastrophic Protection
11) Case-Specific AV
12) Low Cost?
13) Gatekeeping?
14) OON Benefits?
   The value of a plan's catastrophic protection 10 is a function of one benefit design attribute: the out-of-pocket maximum. The net benefit 15 (as described above) reflects a most-likely scenario, based on user responses to the four questions (who is covered, general propensity to consume, events, and conditions). However, a crucial function of insurance (classically, the main function of insurance) is to protect against unforeseen, random events. A person who rarely or never sees a doctor, takes no medications, and anticipates no events or conditions, still might have in the coming year a catastrophic injury or newly presenting illness that would necessitate services costing hundreds of thousands of dollars. In the most-likely case, this person would receive no benefit from spending more in premium for a “richer” plan (i.e. one with lower cost-sharing features). However, in the unforeseen, catastrophic scenario, there is value in spending more for a plan with a lower out-of-pocket maximum.

“Case specific AV” 11 refers to the concept that a plan's “actuarial value” (AV) varies on a case-specific basis. The AV of a product/benefit design is the percentage of covered cost that is paid by the insurer/employer/plan. The remaining percentage is paid by the insured person. Under the Accountable Care Act (ACA), a system of AV characterization was mandated to establish better-informed comparative shopping. In the individual and small-group markets plans of 90%, 80%, 70%, and 60% AV were mandated to be labelled as Platinum, Gold, Silver, or Bronze, respectively. The actuarial values of larger-group plans were/are to be measured by a standardized method (the Federal “AV Calculator”) to establish that a plan is “creditable” for regulatory purposes. These product-level AV's reflect the average AV across the whole population's distribution of claim experiences. However, at the individual level, the effective AV varies based on the individual's circumstances. Consider for illustration a Bronze (60% average AV) plan with an atypically simple design: a $3,000 comprehensive front-end deductible followed by 20% co-insurance to an OOP maximum of $6,000. A person with less than $3,000 in claims pays 100% and the “plan” pays 0%, so the effective AV is 0%. For a person with a million dollars in claims, the insurance company pays all except $6,000, so the effective AV is $994,000/$1,000,000, or 99.4%. AV is a measure of plan quality, and consideration of case-specific AV is one way My Clearview determines best fit for an individual.

“Low cost” 12 as a Value Score 16 modifier recognizes the advantage of a plan that has a near zero, or even net negative cost. Cost to the individual is premium cost, plus OOP cost, minus any employer account donations, if applicable. Independent of variations in the value of services to be consumed (driven by demand elasticity, gatekeeping, and out-of-network benefits), low cost in the most-likely scenario has value to the consumer. Note that most-likely cost can be net negative, i.e. a “money-in-the-bank” scenario can occur if an employer HSA donation exceeds the sum of payroll deduction (employee contribution to premium) and out-of-pocket cost. Incremental value is ascribed to plans with near-zero or net-negative most-likely cost.

Primary-care referral requirements (aka “gatekeeping” 13) has an independent effect on consumers' perceptions of quality of coverage. Gatekeeping's effect on services consumed is on the order of 3%, and this is reflected in the “net benefit” 15 calculation. However, gatekeeping requirements are perceived by most consumers as an inconvenience, and proprietary research suggests that on average consumers would pay 7% more for a plan free of such requirements. Therefore, gatekeeping is an independent value-score modifier.

Similarly, out-of-network (OON) benefits 14 have a value to consumer perception and experience that exceeds its actuarial contribution to claim cost. Actual cost of OON benefits is very low because OON utilization is typically almost nil. Incremental cost can be net negative because higher provider costs can be superseded by higher patient-pay percentages. Nonetheless, OON benefits provide consumers with a peace of mind that supports incremental premiums that exceed incremental claim cost.

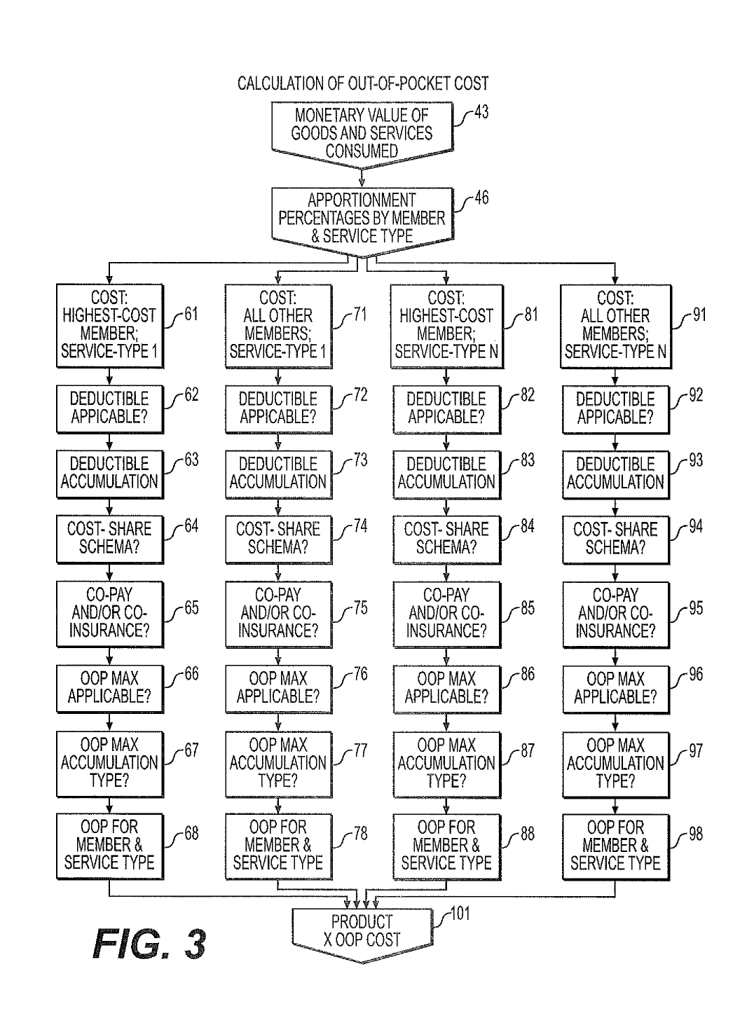

The determination, by the decision support tool, of the net benefit 15, and specifically the services value 8 and out-of-pocket cost 9 are discussed in more detail with reference to FIG. 2 (services value 8) and FIG. 3 (out-of-pocket cost 9).

Projecting the Value of Future Consumption

As discussed above, user responses 1 to the questions below are central to the determination of the value of services projected to be consumed.
31) User Input 1: Who is covered?
32) User Input 2: Propensity to consume
33) User Input 3: Likely Events
34) User Input 4: Likely Conditions

The user chooses one of the five “rate tiers” below to indicate who is covered 31:
Individual-only
Individual plus spouse
Individual plus one child
Individual plus children
Individual plus family

The user then chooses one of five strata to characterize general propensity to consume 32 as:
Very high
High
Average
Low
Very low
   This characterization leads to an initial percentile assignment, which is then modified by any anticipated events or conditions. High-cost events or conditions can supersede a low self-reported general propensity.

Events 33 and conditions 34 are selected to provide a broad representation of major organ systems and mechanisms of disease, presented in terms an average person understands. Specific lists of events and conditions are not inherent to the system's design. These lists are modular and table-driven, as are associated costs, percentile assignments, and rules for interaction. But representative snapshots of event and condition lists are presented below:
Events:
Birth of a child
An inpatient hospital admission
Surgery
5 or more drugs for any covered individual
Any biological Rx (typically $1000+ per month. Examples: Humira, Enbrel, Remicade, Neulasta, Rituxan and Avastin.)
Kidney dialysis
Testing and/or treatment associated with difficulty becoming pregnant
Conditions:
Cancer not in remission
Heart condition requiring medication
Narrowed arteries requiring “blood thinners”
Rheumatoid Arthritis (or any autoimmune disorder or immune system deficiency)
Diabetes or other endocrine (hormonal) disorders
Significant brain or spinal cord disorder
Lung disorder: moderate-to-severe-asthma, emphysema, COPD
Other requiring multiple office visits
Other requiring advanced diagnostics or imaging: e.g. MRI, CT
   Users can check as many or as few (including zero) events 33 and conditions 34 as are applicable. Individual events 33 and conditions 34 are associated with assigned episode-of-care costs and corresponding percentile positions in the respective cost distributions (different distributions for different types of multi-person contracts). When multiple events and/or conditions are reported, the system applies a probability-based algorithm to attribute events/conditions to one person or multiple persons (step 45). This attribution is important for two reasons. First, most contracts employ a method of deductible and OOP-maximum accumulation wherein there is an “individual” deductible and maximum for the highest-cost individual “embedded” within a higher contract-level deductible and maximum. Therefore, $X of collective claims usually results in greater out-of-pocket cost if it is incurred by two or more people, rather than one. Second, when one person has multiple events/conditions, costs increase, but not in an additive manner. For example, multiple problems can be addressed in one office visit or one hospital stay, but certain events and conditions interact with one another in complex ways. However, if events or conditions are borne by different people, then the costs are independent.

In Step 35, a preliminary consumption estimate is generated based on the question responses. This initial estimate is subject to adjustment in subsequent steps. Geographic adjustment is applied at the 3-digit Zip Code level, based on publicly-available data used by the US Government for Medicare reimbursement (steps 37 and 40). As noted above, consumption of services is decreased by “gatekeeping” and increased by out-of-network benefits. These are straight-forward attributes of each benefit design (step 38), and adjustments for their effects are applied in step 41.

In step 42, adjustment is made for “demand elasticity”. Data for event and condition costs (FIG. 3) are based on market-average benefit richness (actuarial value roughly 80%) and population-average utilization. Leaner benefits will result in less utilization and richer benefits will lead to more utilization. For purposes of adjustment for demand elasticity, the present decision tool uses “individualized” AV (as conceptually introduced above) rather than population-average AV.

After the adjustments above, the tool arrives at the Monetary Value of Goods and Services Consumed (step 43). This is one of the four components of net value depicted in steps 8 and 141. As such, step 43 feeds forward as a key component of ordinal ranking and Value Score.

Projecting Out-of-Pocket Cost

Out-of-pocket cost as shown in FIG. 3 is a function of projected cost and benefit-design detail. Most plans have different patient-pay (out-of-pocket) features for different types of service. Therefore, it is necessary to separate overall utilization into various service types (e.g. the twenty-seven services listed above).

In step 43 contract-level cost is projected. In steps 36, 44, 45, and 46 all from FIG. 2, the total cost is apportioned into the various service types. Apportionment is driven primarily by percentile (step 36). In step 44, the distribution of claimants is stratified into numerous, in one example eighty-four, categories, each with its own empirically-based pattern of apportionment by type of service. Lower-percentile strata will have a high percentage of cost in office visit and prescription drug categories, and higher percentile strata will have high percentages in hospital and surgery categories. In step 45, the system applies a probability-based algorithm to attribute events/conditions to one person or multiple persons (step 45). Percentage apportionments by member and type of service, having been derived in step 45, feed forward to calculate projected out-of-pocket cost.

Steps 61, 71, 81, and 91 depict total cost 43 apportioned by member and service-type percentages 46. The member dimension has two degrees of freedom, characterized as highest-cost member and all-others. This separation is necessary to model “embedded” forms of deductible and OOP-maximum accumulation. Further separation for contracts with more than two members would not contribute further to precise OOP calculation. Note that the service-type dimension has twenty-seven categories, but is represented in the flowcharts in abbreviated form as 1-N. A current working example of the decision tool has fifty-four cells that are represented in the flowcharts in the four cells (61, 71, 81, and 91). For each subset of apportioned cost, a contribution to total OOP cost is calculated (e.g. in steps 62-68). For each service type, a deductible might or might not apply (62), and that deductible could be either “embedded” or “non-embedded” (63). Cost-sharing beyond the deductible might be according to any of four “schemas” (64 and 65):

Co-payment

Co-Insurance

Combination of Co-Insurance and Co-payment

Greater of Co-Insurance or Co-payment up to a per-unit maximum

Out-of-pocket amounts (deductible plus schema-dependent) for a particular service type might or might not accrue to an OOP maximum 66), which might accumulate in an “embedded” or “non-embedded” manner 67.

As the “silo” OOP amounts are developed (represented in steps 68, 78, 88, and 98) member-level and contract-level deductible accumulations are modeled, and deductibles cease to have impact when they are met. Similarly, the accumulation of member-level OOP maximums is modeled and applied when met. Finally, the subset OOP amounts are summed in step 101, and the contract-level OOP-maximum is applied. The OOP amount in step 101 is one of the four major components of net cost and ordinal ranking, and it feeds forward for Value Score calculation.

Derivation of Value Scores

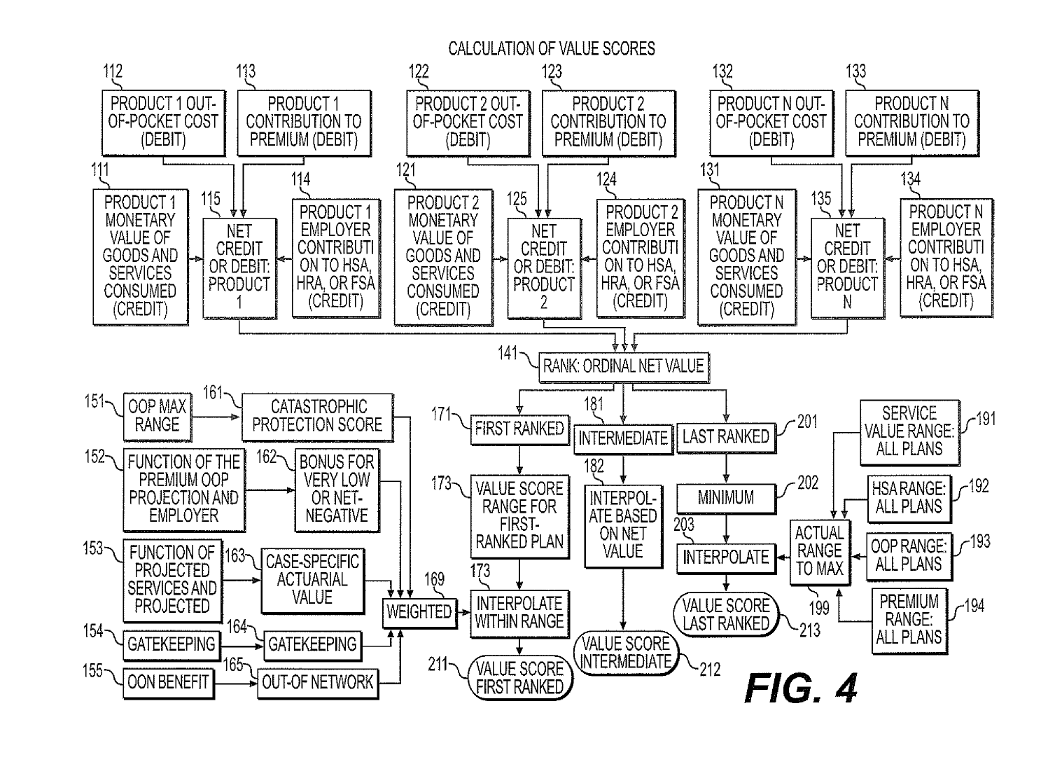

The calculation of Value Scores is illustrated in FIG. 4. As previously discussed, the net of benefits (services value plus HSA) and costs (premium plus OOP) is calculated for each product. The four components are represented in steps 111-114 for an example medical insurance Product 1, steps 121-124 for example Product 2, and steps 131-134 for example Product N. In practice, there could be scores of medical insurance products in a public exchange situation, though there usually are only three or four in an employer-based situation. For each product, the four components are summed (debit values being negative numbers) in steps 115, 125, and 135. Then in step 141 the ordinal ranking from highest to lowest net benefit is determined.

Beyond the ordinal ranking, Value Scores on a 100-point scale are assigned. The purpose is to convey to the user more information than is conveyed by ranking alone. For example: is the highest-ranked plan a great fit for the individual, or merely a better fit than the other options? As discussed in the Overview section above, and with reference to FIG. 1, modifying factors are:
10) Catastrophic Protection
11) Case-Specific AV
12) Low Cost?
13) Gatekeeping?
14) OON Benefits?
   These modifying factors are also represented in FIG. 4 as:
161) Catastrophic Protection Score
162) Bonus for very low or net-negative cost?
163) Case-Specific Actuarial Value
164) Gatekeeping penalty?
165) Out-of-network bonus?
   A weighted average of the above factors determines where (step 173) the first-ranked product falls within the allowed range of scores for a first-ranked plan (step 172). The result is 211, the Value Score for the first-ranked plan.

Value Scores for the lowest-ranked plan 213 and for intermediate-ranked plans 212 convey to the user whether values are closely clustered or far apart. Having established the Value Score for the highest-ranked plan 211, the range of value score results is established by assigning a score to the lowest-ranked plan 213. Each of the four components of net benefit has an independent range of results (steps 191-194). The sum of these is the denominator of an interpolation factor (step 199), the numerator of which is the net-benefit difference between best-plan and worst plan. A minimum score is administratively assigned (step 202), and interpolation (step 203) determines where in the allowable range of scores the lowest-ranked plan will fall. The result is the lowest rank Value Score in step 213. Value Scores for intermediate-ranked plans (step 212) are determined by interpolation based on relative net value (step 182).

The present decision-support tool can be embodied in the form of methods and apparatus for practicing those methods. The present invention can also be embodied in the form of program code embodied in tangible media, such as CD-ROMs, hard drives, or any other machine-readable storage medium, wherein, when the program code is loaded into and executed by a machine, such as a computer, laptop, tablet or mobile device, the machine becomes an apparatus for practicing the invention. The present invention can also be embodied in the form of program code, for example, whether stored in a storage medium, loaded into and/or executed by a machine, or transmitted over some transmission medium, such as over electrical wiring or cabling, through fiber optics, or via electromagnetic radiation, wherein, when the program code is loaded into and executed by a machine, such as a computer, the machine becomes an apparatus for practicing the invention. When implemented on a general-purpose processor, the program code segments combine with the processor to provide a unique device that operates analogously to specific logic circuits.

Example User Experience

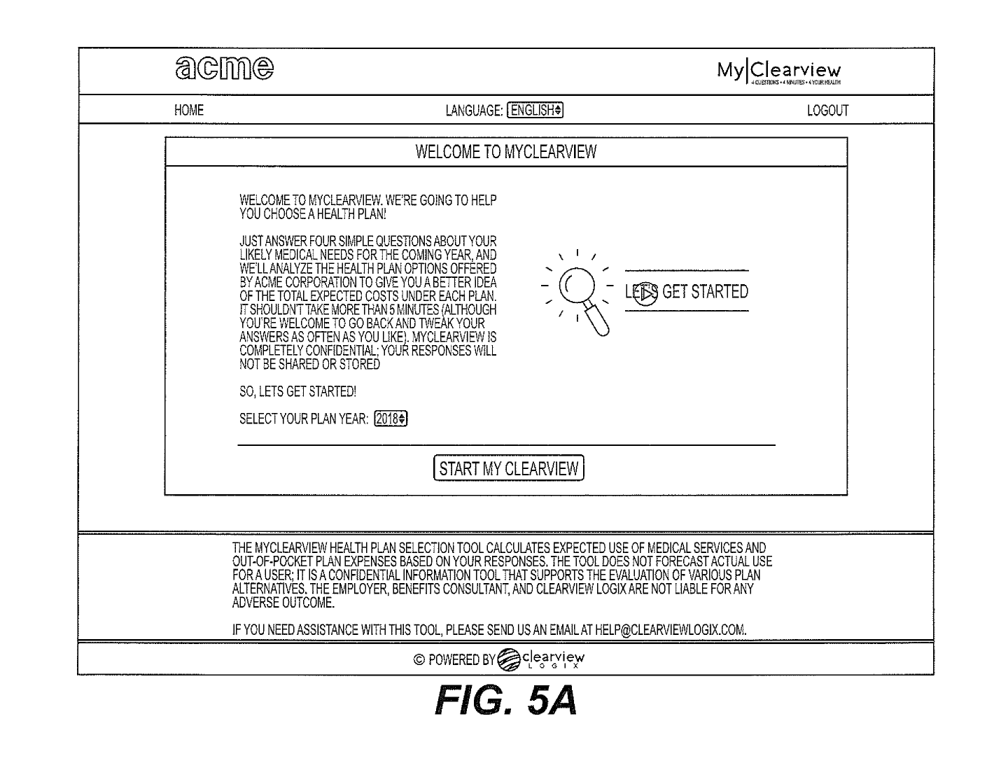

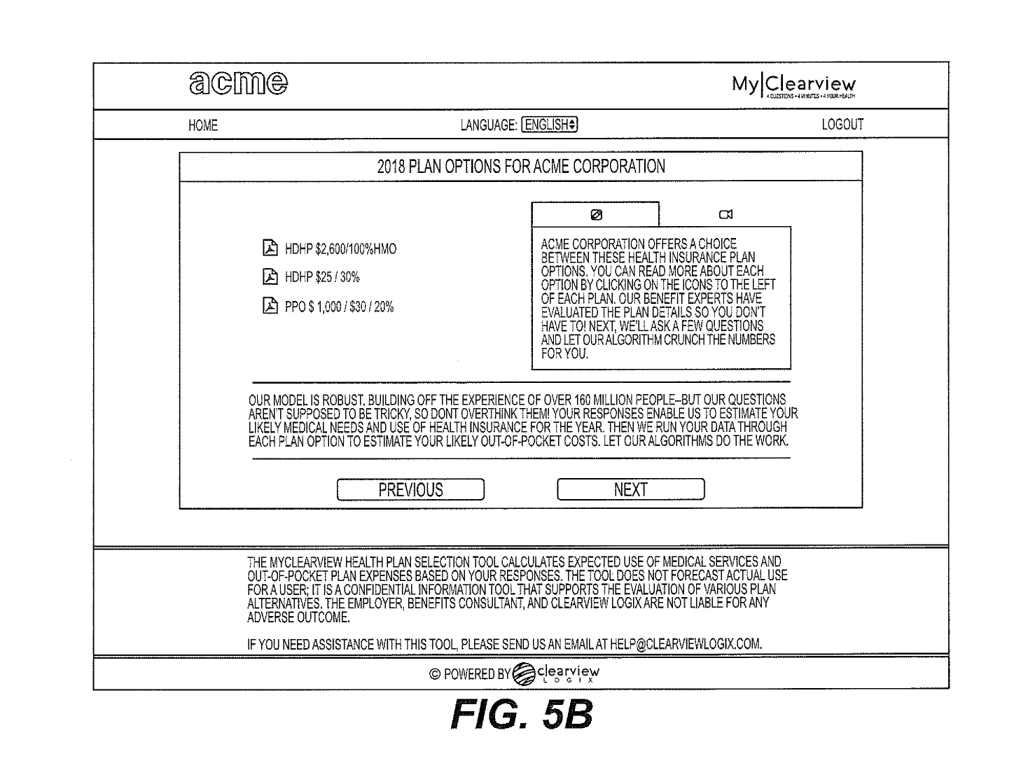

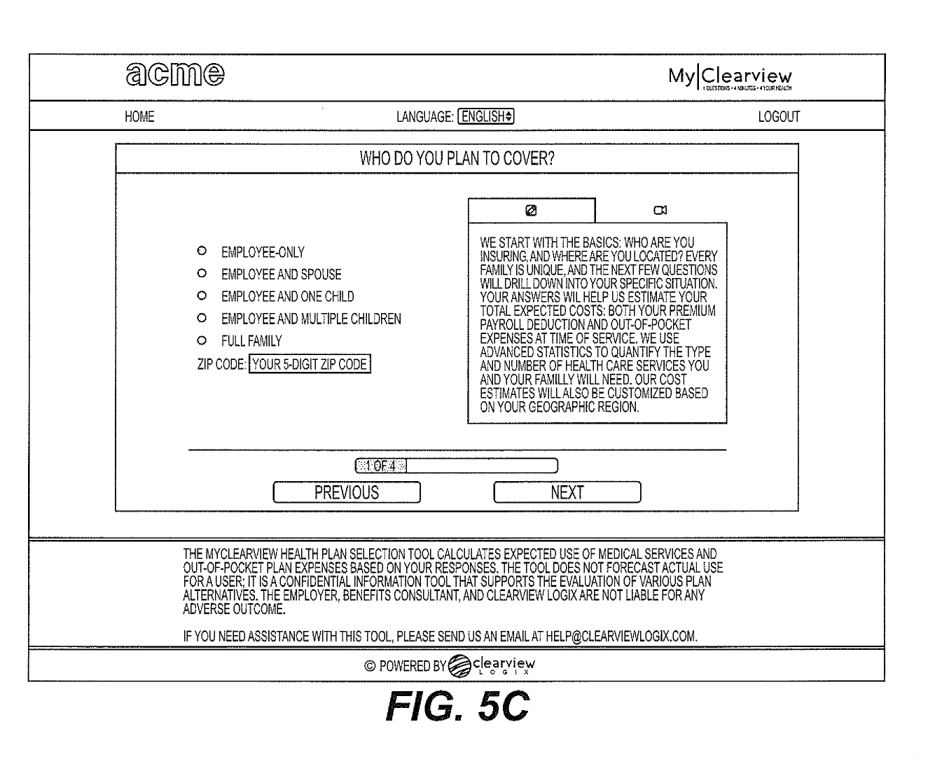

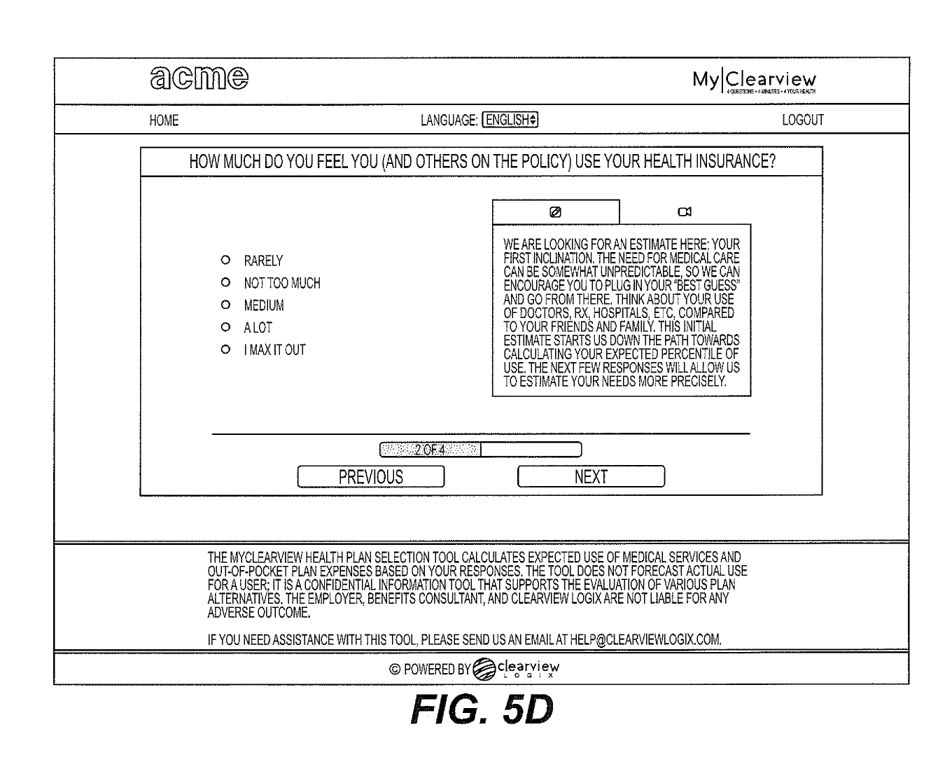

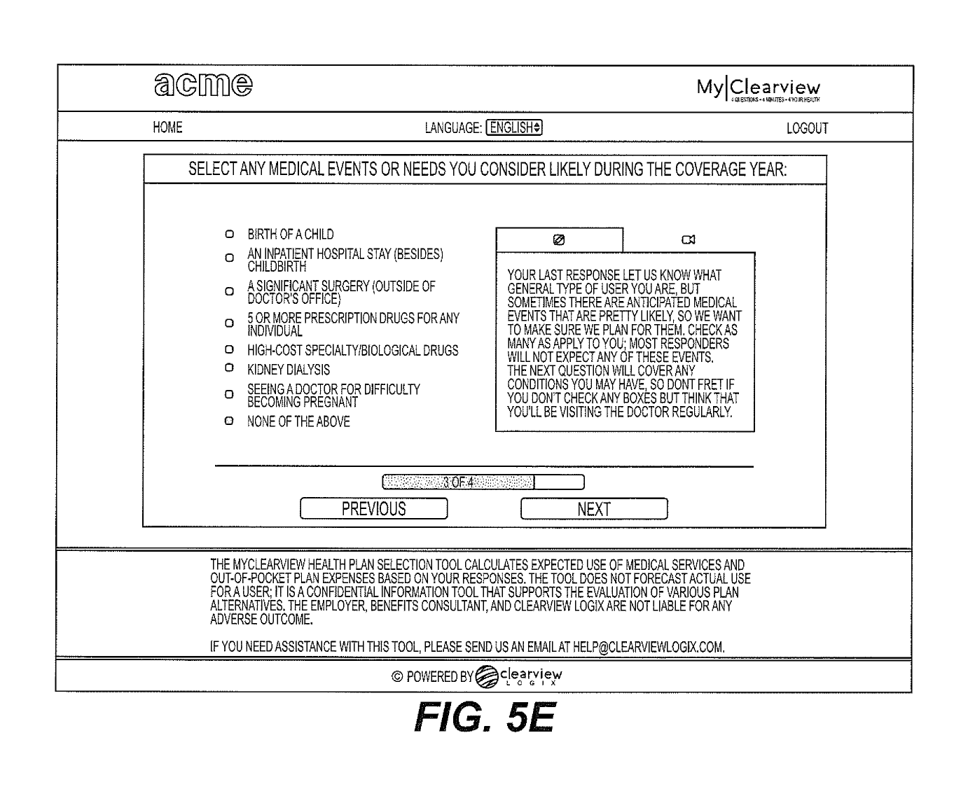

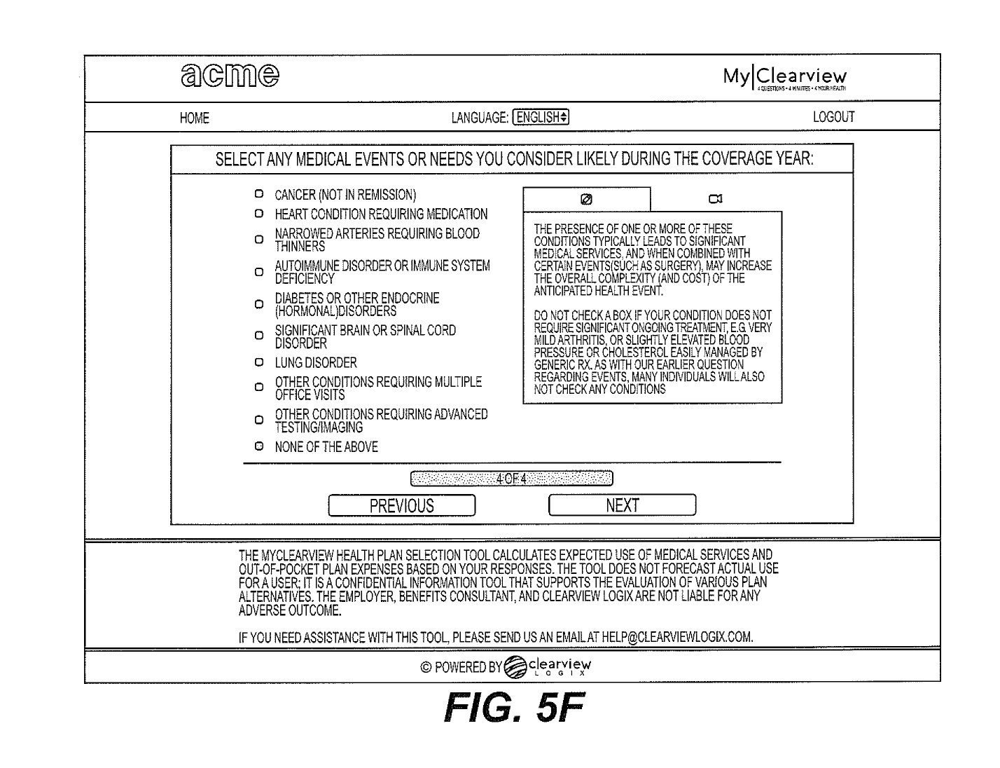

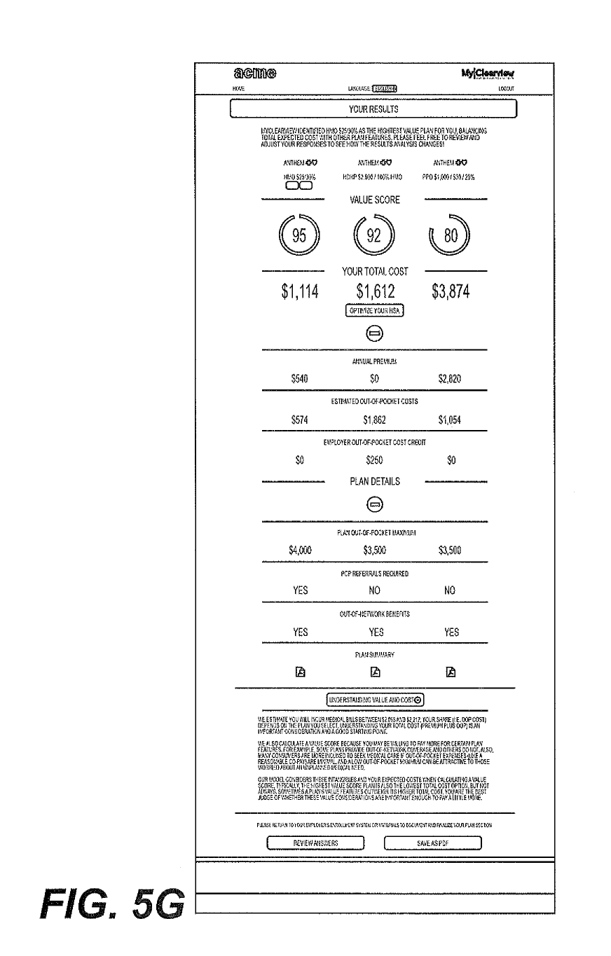

FIGS. 5A-5G illustrate an example of the user experience by showing an example of user interfaces in a hypothetical user plan. For the simplicity and brevity preferred by most users, ordinal display of Value Scores is front and center in the results. However, more inquisitive users electively can uncover detail behind the rankings and scores. The user experience is summarized by the following sequence of exemplary “screenshots”:

FIG. 5A is a user interface landing page that explains the tool and the intended use of the decision tool. It should be noted that the decision tool in FIGS. 5A-5G is referred to in these examples by its trademark MyClearview. The example employer in this hypothetical is Acme.

FIG. 5B is a user interface that shows an example of a list of the medical health insurance products available to this example user. Of course, there may be more or fewer plan options available to the user of the decision tool.

FIG. 5C is the first of four questions presented to the user of the tool. In this step, the first question is directed to who the plan is expected to cover. There are four basic questions that will be presented to a user. These questions may be presented in any order and may be answered in any order.

FIG. 5D is addressed to the question of the user's inclination with respect to the propensity to use a health insurance plan.

FIG. 5E is addressed to the question of the user's opinion with respect to medical events that the user expects are likely in the coming year.

FIG. 5F is addressed to the question of a user's opinion with respect to medical conditions that the user believes likely in the next year.

And finally, FIG. 5G is the results page that shows the decision tool's analysis and the value scores assigned to the insurance plan options. The user may follow of ignore the value scores as they wish, but the decision tools provides useful guidance.

Alternative Decision Tools The additional medical plan features of interest to consumers include network participation detail and drug formulary detail. If/when adequate industry data sources become available, these issues may be incorporated into versions of the decision tool. However, any increase in content would need to be weighed against the goal of a brief (less than 4-minute average), streamlined user experience that encourages high voluntary use rates (greater than 50%).

Where better network participation data is available, it might be used in at least two different ways. One would be a quantified score reflecting overall doctor and hospital participation rates on local, regional, state, and national levels. The issue with such a system is that the importance of network statistics to any particular user is highly idiosyncratic. Many people are interested only in the participation status of one or two doctors or doctor groups. The ability to perform a physician-specific or hospital-specific lookup might be a desirable enhancement for many users. But in any event, network-related inquiries might be elective and tangential rather than mandatory mainstream, in order to maintain the desired brevity of the user experience. Similarly, some users might have an interest in the formulary status (i.e. “preferred” second tier vs. “non-preferred” third tier) of one or several specific drugs. Where comprehensive, robust, and reliable formulary data is available, some users would appreciate a specific lookup capability. As with network inquiries, a drug lookup feature would likely be an elective tangent to preserve the desired brevity and simplicity of the user experience.

Also, the decision tool allows for the generation of a heat map based on actual usage in a particular group. The heat map involves testing a statistically valid sample of user response scenarios for a range of results covering very low to very high users of medical services, across all family tiers. The results of a heat map may highlight, for example, an unintended misalignment in employee premium subsidies between two products. The heat map can give an employer or exchange the information needed to present recommendations for changes to the product mix and premium contributions by employees. A heat map can provide vital information and confirmation of expected results for the employer and broker/consultant before employees experience it.

Other embodiments of the present invention will be apparent to those skilled in the art from consideration of the specification. It is intended that the specification and figures be considered as exemplary only, with a true scope and spirit of the invention being indicated by the claims.

That which is claimed is:

1. A method of presenting to a user a ranking of available medical insurance options comprising the steps of:
providing a processor for storing historical data regarding available medical insurance policies and for calculating estimated future medical insurance costs,
administratively inputting and storing in the processor information regarding medical and prescription drug costs by event and condition, by percentile and service type and by geographic location,
administratively inputting and storing in the processor group-specific data regarding benefit design data,
inputting by a user into the processor the user-estimated medical needs,
calculating by the processor an estimated cost of goods and services to be consumed by the user, an estimated out-of-pocket cost to the user, and a net benefit to the user, and
using the net benefit calculation plus other quality-of-coverage attributes, calculating and presenting to the user value scores with respect to each medical insurance option available to the user in the group-specific plan.

2. A method of presenting to a user a ranking of available medical insurance options as described in claim 1, wherein the value scores are characterized as a single relative value score.

3. A method of presenting to a user a ranking of available medical insurance options as described in claim 1, wherein the user-estimated medical needs input by the user do not include user-level medical or prescription drug claim data.

4. A method of presenting to a user a ranking of available medical insurance options as described in claim 1,
wherein the net benefit calculation is the net of two positives including monetary value of covered services and employer contribution to a user HSA/HRA/FSA account, and two negatives including employee contribution to premium and estimated out of pocket cost.

5. A method of presenting to a user a ranking of available medical insurance options as described in claim 4, wherein the out of pocket estimate is an apportionment of projected cost over multiple service types.

6. A method of presenting to a user a ranking of available medical insurance options as described in claim 5, wherein the apportionment includes at least ten service types.

7. A method of presenting to a user a ranking of available medical insurance options as described in claim 5, wherein the apportionment by service type is based on the percentile of the user's projected cost.

8. A method of presenting to a user a ranking of available medical insurance options as described in claim 4, wherein the apportionment is the projected cost by member within the contract.

9. A method of presenting to a user a ranking of available medical insurance options as described in claim 7, wherein the percentiles are based on distributions from individual-only contracts.

10. A method of presenting to a user a ranking of available medical insurance options as described in claim 7, wherein percentiles for multi-person contracts are derived from Monte Carlo simulations.

11. A method of presenting to a user a ranking of available medical insurance options as described in claim 4, wherein the benefit design model is based on plan-level data including gatekeeping, out-of-network benefits, and accumulation method; tier-level data including deductibles, maxima, and contributions; and service-level data including out-of-pocket schema and applicability of deductibles and maxima.

12. A method of presenting to a user a ranking of available medical insurance options as described in claim 11, wherein out-of-pocket schema include co-payments, co-insurance, combined co-payments and co-insurance, and greater of co-payment or co-insurance up to a per-service maximum.

13. A method of presenting to a user a ranking of available medical insurance options as described in claim 1, wherein the value score calculation includes quality attributes that do not affect out-of-pocket cost including catastrophic protection, gatekeeping, case-specific actuarial value, out-of-network coverage, and negative or near-zero net cost.

14. A method of presenting to a user a ranking of available medical insurance options as described in claim 1, wherein the user input is based on only four questions asked.

15. A method of presenting to a user a ranking of available medical insurance options as described in claim 14, wherein the four questions are who is covered, general propensity to consume medical services, anticipated medical events, and anticipated medical conditions.

16. A method of presenting to a user a ranking of available medical insurance options as described in claim 1, also comprising the step of calculating and displaying to an employer a percentile-based heat map regarding a summary of multiple user contribution strategy and benefit design.
